(19) United States
(12) Patent Application Publication
(10) Pub. No.: US 2015/0258127 A1
(43) Pub. Date: Sep. 17, 2015

(54) METHODS FOR PREVENTING ANTIPHOSPHOLIPID SYNDROME (APS)

(71) Applicants: INSERM (INSTITUT NATIONAL DE LA SANTÉ ET DE LA RECHERCHE MÉDICALE), Paris (FR); UNIVERSITE PARIS DESCARTES, Paris (FR)

(72) Inventors: Fabiola Terzi, Paris (FR); Guillaume Canaud, Paris (FR); Frank Bienaime, Paris (FR)

(21) Appl. No.: 14/439,266

(22) PCT Filed: Oct. 31, 2013
(86) PCT No.: PCT/EP2013/072840

§ 371 Date: Apr. 29, 2015

(30) Foreign Application Priority Data
Oct. 31, 2012 (EP) 12306365.3

(51) Int. Cl. A61K 31/685; A61K 31/635; A61K 31/496; A61K 31/4745; A61K 45/06; A61K 31/415; A61K 31/4439; A61K 31/436; A61K 31/675; A61K 31/519; A61K 31/5377; A61K 31/437

(52) U.S. Cl. CPC A61K 31/685; A61K 31/5377; A61K 31/635; A61K 31/496; A61K 31/4745; A61K 31/437; A61K 31/415; A61K 31/4439; A61K 31/436; A61K 31/675; A61K 31/519; A61K 45/06

(57) ABSTRACT

The present invention relates to the prevention or treatment of antiphospholipid syndrome (APS) in a patient in need thereof (e.g. patients affected with primary APS, a secondary APS, a catastrophic APS (CAPS) or a transplant recipient with antiphospholipid antibodies (APA)). The present invention also relates to the prevention APS-related vascular lesions in said a patient in need thereof. The present invention further relates to PI3K-AKT-mTOR pathway inhibitor for use in inhibiting endothelial m TORC activation triggered by APA in a patient in need thereof.

### FIELD OF THE INVENTION

The present invention relates to the prevention or treatment of antiphospholipid syndrome (APS). The present invention also relates to the prevention APS-related vascular lesions in a patient in need thereof (e.g. patients affected with primary APS, a secondary APS, a catastrophic APS (CAPS) or a transplant recipient with antiphospholipid antibodies (APA)). The present invention further relates to the inhibition of endothelial mTORC activation triggered by APA in a patient in need thereof.

### BACKGROUND OF THE INVENTION

Antiphospholipid syndrome (APS) is an autoimmune disease characterized by the presence of circulating antiphospholipid antibodies (APA also referred as aPL) that cause arterial, venous and small vessels thrombosis and/or obstetrical complications consisting in pregnancy loss or preterm birth due to pre-eclampsia or placental insufficiencyl. APA are a family of autoantibodies that recognize various phospholipids and plasma proteins with affinity for anionic cell surface phospholipids. There are three main types of APA: lupus anticoagulant (LA), anti-cardiolipin (aCL) and anti-β2 glycoprotein I antibodies (anti-β2GPI)¹. APS is observed either isolated or in association with in a number of autoimmune disorders, i.e. systemic lupus erythematosus (SLE).

APS is considered as the most frequent cause for acquired thrombophilia and is associated with high morbidity and mortality¹. APS account for 20% of the stroke in young patients. In addition, APS represents a major adverse prognostic factor in patients with SLE². The main consequence of the APS is thrombotic complications³, and so far, the only treatment, which has been shown to reduce the vascular complications in APS patients, is permanent anticoagulation. However, this regimen does not completely prevent the recurrence of thrombosis in high risks patients and is associated with an increase incidence of bleeding.

Although thrombosis is considered as the key feature of the vascular disease in APS, chronic arterial and arteriolar lesions have been frequently associated. These lesions consist mainly in thickening of the intima and the media and are often associated with increased cellularity of the two layers⁴⁻¹¹. These lesions have been particularly well characterized in the kidney and called APS-nephropathy (APSN). These vascular changes lead to progressive fibrosis that ultimately results in end-stage renal failure (ESRF)¹²⁻¹⁴. Moreover, it has been reported that kidney transplant recipients with APA are at greater risk to develop thrombotic complication¹⁵⁻¹⁹. In addition to thrombotic complication, it has been observed that these patients developed typical features of APSN recurrence on the allograft¹⁵. These lesions led to a fast decline of the measured glomerular filtration rate (mGFR).

To date the effort made to elucidate the pathogenesis of APS have focused on the mechanisms of thrombosis formation whereas the pathophysiological processes responsible for the chronic vascular changes associated with APS have not been investigated. In that regard, a better understanding of APSN pathogeny could represent an important milestone to elaborate therapeutic strategies limiting the chronic vascular alterations in APS. The pathophysiology of these lesions is unknown and efficient therapeutic strategy are lacking.

mTORC kinase is a central node signalling pathways that regulate cellular growth, proliferation and survival. mTOR is a component of two functionally distinct complexes. mTOR complex 1 (mTORC1) stimulates ribosome biogenesis and protein translation by phosphorylating S6 kinase while in turn activates S6 ribosomal protein (S6RP), and 4E-BP1 protein (4EBP1). mTOR complex 2 (mTORC2) promotes survival, proliferation or migration depending on the cellular context, through AKT phosphorylation on Ser⁴⁷³. An important and complex cross-regulation exists between mTORC1 and mTORC2. Indeed, the activation of AKT by mTORC2 stimulates mTORC1, whereas mTORC1 reduces mTORC2 activation²⁰. mTORC has been shown to play an important role in the vascular narrowing secondary to mechanical endothelial injury in both experimental models and patients undergoing arterial angioplasty notably by promoting vascular smooth muscle cells (VSMC) proliferation in the media²¹⁻²⁴. Indeed the mTORC inhibitor sirolimus is now currently used to prevent reactive arterial stenosis after coronary artery stenting.

However, the activation of the mTOR pathway in endothelial cells by APA leading to the vascular lesions of APSN has never been studied nor even suggested until now.

### SUMMARY OF THE INVENTION

In a first aspect, the present invention also relates to a PI3K-AKT-mTOR pathway inhibitor for use in the prevention of APS-related vascular lesions in a patient in need thereof.

In a second aspect, the present invention also relates to a PI3K-AKT-mTOR pathway inhibitor for use in inhibiting endothelial mTORC activation triggered antiphospholipid antibodies (APA) in a patient in need thereof.

In a third aspect, the present invention further relates to a pharmaceutical composition for use in the prevention of APS-related vascular lesions comprising a PI3K-AKT-mTOR pathway inhibitor and a pharmaceutically acceptable carrier.

In still another aspect, the present invention relates to a kit comprising at least two PI3K-AKT-mTOR pathway inhibitors, as a combined preparation for simultaneous, separate or sequential use in the prevention of APS-related vascular lesions.

### DETAILED DESCRIPTION OF THE INVENTION

The present invention is based on the vascular activation of both mTORC1 and 2 pathways in APSN as well as in others critical arterial beds in patients with severe APS. Remarkably, this activation concerned selectively the endothelial cells but correlated with proliferation of both endothelial and smooth muscle cells and, more importantly, with vascular lesions. Thus, the inventors demonstrated for the first time the crucial role played by endothelial mTORC pathway activation in the development of the fibrous intimal hyperplasia in APS patients. Mechanistically purified IgG from patients with APS activate both mTORC1 and mTORC2 in cultured endothelial cells in a complement independent manner. Briefly, these antiphospholipid IgG were collected from 12 different patients. Among these patients, 7 underwent a kidney biopsy that revealed the presence of APS nephropathy with the characteristic vascular lesions. All the tested antiphospholipid IgG were able to activate the mTORC pathway in vitro and in vivo and the intensity of activation correlates with the titers of antibodies. Remarkably, as disclosed herein, mTORC inhibition in kidney transplant recipient with recurrent APSN was associated with a reduction of the severity of vascular lesions and with a marked improvement of allograft survival.

Accordingly, the inventors demonstrated for the first a beneficial effect of rapamycin in preventing vascular lesions during APS. Remarkably, if sirolimus administration increased the allograft survival rate from 8 to 70% in transplant recipients with antiphospholipid antibodies, it did not improve the allograft outcome in the control group at least up to 144 months post transplantation Notably, sirolimus administration was also associated with a dramatic increase (12% versus 70%) of death-censored allograft survival in Tx aPL+ recipients exclusively, consistent with a direct impact of the treatment on renal lesions.

### DEFINITIONS

Throughout the specification, several terms are employed and are defined in the following paragraphs.

The terms “antiphospholipid syndrome” or “antiphospholipid antibody syndrome” (APS), often also Hughes syndrome, refer to an autoimmune disease characterized by the presence of circulating antiphospholipid antibodies (APA also referred as aPL) that cause arterial, venous and small vessels thrombosis and/or obstetrical complications consisting in pregnancy loss or preterm birth due to pre-eclampsia or placental insufficiency. In particular, the disease is characterised by antibodies against lupus anticoagulant (LA), cardiolipin (anti-cardiolipin antibodies) and-β2 glycoprotein I (anti-β2GPI). The term “primary antiphospholipid syndrome” is used when APS occurs in the absence of any other related disease. APS however also occurs in the context of other autoimmune diseases, such as systemic lupus erythematosus (SLE), in which case the term “secondary antiphospholipid syndrome” is used. In rare cases, APS leads to rapid organ failure due to generalised thrombosis; this is termed “catastrophic antiphospholipid syndrome” (CAPS) and is associated with a high risk of death.

As used herein, the term “phosphatidylinositol 3-kinase” (PI3K) is well known in the art and refers to a family of lipid kinases consists of at least eight proteins with shared sequence homology within their kinase domains, but with different substrate specificities and modes of regulation. The best known members are the four Class I PI3K isoforms (a, (3, 6, and y), which convert PIP2 to PIP3.

As used herein, the term “AKT” (also known as protein kinase B or PKB) is well known in the art and refers to a protein serine/threonine kinase that was first discovered as an oncogene transduced by the acute transforming retrovirus (AKT-8).

As used herein, the term “mammalian target of rapamycin” (mTOR) is well known in the art and refers to a multidomain serine/threonine kinase, which has a catalytic domain that has homology with the PI3K family of protein kinases. mTOR (also known as FK506 binding protein 12-rapamycin associated protein 1 or FRAP) is an important signaling intermediate molecule downstream of the PI3K/AKT pathway that inhibits apoptosis and functions as a sensor of nutrient and energy levels and redox status.

As used herein, the term “patient” refers to an animal, preferably to a mammal, even more preferably to a human, including adult and child. However, the term “subject” can also refer to non-human animals, in particular mammals such as cats, horses, and non-human primates, among others, that are in need of treatment.

### Therapeutic Methods and Uses

The present invention provides methods and compositions (such as pharmaceutical compositions) for preventing or treating antiphospholipid syndrome (APS) in a patient in need thereof. The present invention also provides methods and compositions for inhibiting or preventing APS-related vascular lesions in a patient in need thereof.

According to a first aspect, the present invention relates to a phosphatidylinositide 3-kinase (PI3K)-AKT-mammalian target of rapamycin (mTOR) pathway inhibitor for use in the prevention or the treatment of antiphospholipid syndrome (APS) in a patient in need thereof.

In a second aspect, the present invention also relates to a PI3K-AKT-mTOR pathway inhibitor for use in the prevention of APS-related vascular lesions in a patient in need thereof.

In one embodiment, the APS-related vascular lesions are APS-nephropathy (APSN).

Lesions related to APS-nephropathy are well known in the art and may be quantified according to the criteria as described¹². For each biopsy, the number of vessels displaying fibrous intimal hyperplasia may be counted in all the fields of the section and expressed as the number of damaged vessels for the total number of vascular sections.

In another aspect, the present invention further relates to a PI3K-AKT-mTOR pathway inhibitor for use in reducing or inhibiting endothelial mTOR activation triggered by antiphospholipid antibodies (APA) in a patient in need thereof.

In still another aspect, the present invention further relates to a PI3K-AKT-mTOR pathway inhibitor for use in preventing graft rejection and/or preserving graft function in a patient in need thereof.

In one embodiment, the patient in need thereof is affected with a primary APS, a secondary APS, a catastrophic APS (CAPS) or is a transplant recipient with antiphospholipid antibodies (APA). In one embodiment, the patient in need thereof is a patient with APA. In one embodiment, the patient in need thereof is a patient with APA with thrombotic events. In one embodiment, the transplant recipient with APA is selected from the group consisting of a kidney transplant recipient, lung transplant recipient, heart transplant recipient and liver transplant recipient. In one particular embodiment, the transplant recipient with APA is a kidney transplant recipient.

Such inhibitors of PI3K-AKT-mTOR pathway may be selected among small molecule, siRNA, shRNA, anti-sense DNA and the like.

In one embodiment, such inhibitor of PI3K-AKT-mTOR pathway is selected from the group consisting of siRNA, shRNA, anti-sense oligonucleotides and ribozymes.

Small inhibitory RNAs (siRNAs) can function as inhibitors of gene expression of a component of PI3K-AKT-mTOR pathway. For example, gene expression of PI3K, AKT or a member of mTORC complex can be reduced by contacting a subject or cell with a small double stranded RNA (dsRNA), or a vector or construct causing the production of a small double stranded RNA, such that said gene expression of PI3K, AKT or a member of mTORC complex is specifically inhibited (i.e. RNA interference or RNAi). Methods for selecting an appropriate dsRNA or dsRNA-encoding vector are well known in the art for genes whose sequence is known (e.g. see for example Tuschl, T. et al. Genes Dev. 1999 Dec. 15; 13(24):3191-7; Elbashir, S. M. et al Nature. 2001 May 24; 411(6836):494-8; Hannon, G J. Nature. 2002 Jul. 11; 418(6894):244-51); McManus, M T. et al. J Immunol 169, 5754-5760 (2002); Brummelkamp, T R. et al. Science. 2002 Apr. 19; 296(5567):550-3; U.S. Pat. Nos. 6,573,099 and 6,506,559; and International Patent Publication Nos. WO 01/36646, WO 99/32619, and WO 01/68836). All means and methods which result in a decrease in PI3K gene expression, AKT gene expression or in a member of mTOR complex gene expression, in particular by taking advantage of specific siRNAs (i.e siRNAs that target specifically mRNA) may be used in the present invention. Methods for generating and preparing siRNA(s) as well as method for inhibiting the expression of a target gene are also described for example in WO02/055693.

siRNAs or related nucleic acids useful as inhibitors of PI3K, AKT or a member of mTOR complex gene expression, such as anti-sense oligonucleotides can be prepared by known methods. These include techniques for chemical synthesis such as, e.g., by solid phase phosphoramadite chemical synthesis. Alternatively, anti-sense RNA molecules can be generated by in vitro or in vivo transcription of DNA sequences encoding the RNA molecule.

Such DNA sequences can be incorporated into a wide variety of vectors that incorporate suitable RNA polymerase promoters such as the T7 or SP6 polymerase promoters. Various modifications to the oligonucleotides of the invention can be introduced as a means of increasing intracellular stability and half-life. Possible modifications include but are not limited to the addition of flanking sequences of ribonucleotides or deoxyribonucleotides to the 5′ and/or 3′ ends of the molecule, or the use of phosphorothioate or 2′-O-methyl rather than phosphodiesterase linkages within the oligonucleotide backbone. Those modification includes the use of nucleosides with modified sugar moieties, including without limitation, 5′-vinyl, 5′-methyl (R or S), 4′-S, 2′-F, 2′-OCH3 and 2′-O(CH2)2OCH3 substituent groups. The substituent at the 2′ position can also be selected from allyl, amino, azido, thio, O-allyl, O—C1-C10 alkyl, OCF3, O(CH2)2SCH3, O(CH2)2-O—N(Rm)(Rn), and O—CH2-C(═O)—N(Rm)(Rn), where each Rm and Rn is, independently, H or substituted or unsubstituted C1-C10 alkyl.

Antisense oligonucleotides and siRNAs or related nucleic acids useful as inhibitors of PI3K-AKT-mTOR pathway may be delivered in vivo alone or in association with a vector. In its broadest sense, a “vector” is any vehicle capable of facilitating the transfer of the antisense oligonucleotide or siRNA or related nucleic acids to the target cells, preferably those with deficient expression of SMN gene, such as muscular cells. Preferably, the vector transports the nucleic acid to cells with reduced degradation relative to the extent of degradation that would result in the absence of the vector. In general, the vectors useful in the invention include, but are not limited to, plasmids, phagemids, viruses, transposon-based vectors or other vehicles derived from viral or bacterial sources that have been manipulated by the insertion or incorporation of the antisense oligonucleotide or siRNA or related nucleic acid sequences. Viral vectors are a preferred type of vector and include, but are not limited to, nucleic acid sequences from the following viruses: retrovirus, such as moloney murine leukemia virus, harvey murine sarcoma virus, murine mammary tumor virus, and rouse sarcoma virus; adenovirus, adeno-associated virus; SV40-type viruses; polyoma viruses; Epstein-Barr viruses; papilloma viruses; herpes virus; vaccinia virus; polio virus; and RNA virus such as a retrovirus. One can readily employ other vectors not named but known to the art.

Preferred viral vectors are based on non-cytopathic eukaryotic viruses in which non-essential genes have been replaced with the gene of interest. Non-cytopathic viruses include retroviruses (e.g., lentivirus), the life cycle of which involves reverse transcription of genomic viral RNA into DNA with subsequent proviral integration into host cellular DNA. Retroviruses have been approved for human gene therapy trials. Most useful are those retroviruses that are replication-deficient (i.e., capable of directing synthesis of the desired proteins, but incapable of manufacturing an infectious particle). Such genetically altered retroviral expression vectors have general utility for the high-efficiency transduction of genes in vivo. Standard protocols for producing replication-deficient retroviruses (including the steps of incorporation of exogenous genetic material into a plasmid, transfection of a packaging cell lined with plasmid, production of recombinant retroviruses by the packaging cell line, collection of viral particles from tissue culture media, and infection of the target cells with viral particles) are provided in Varmus, Harold; Coffin, John M.; Hughes, Stephen H., ed (1997). “Principles of Retroviral Vector Design”. Retroviruses. Plainview, N.Y.: Cold Spring Harbor Laboratory Press. ISBN 0-87969-571-4.

Preferred viruses for certain applications are the adeno-viruses and adeno-associated viruses or retroviral vectors such as lentiviruses, which are double-stranded DNA viruses that have already been approved for human use in gene therapy. Examples of such viral vectors includes vectors originated from retroviruses such as HW (Human Immunodeficiency Virus), MLV (Murine Leukemia Virus), ASLV (Avian Sarcoma/Leukosis Virus), SNV (Spleen Necrosis Virus), RSV (Rous Sarcoma Virus), MMTV (Mouse Mammary Tumor Virus), etc, lentivirus, Adeno-associated viruses, and Herpes Simplex Virus, but are not limited to. These viral vectors can be engineered to be replication deficient and is capable of infecting a wide range of cell types and species. It further has advantages such as, heat and lipid solvent stability; high transduction frequencies in cells of diverse lineages, including hematopoietic cells; and lack of superinfection inhibition thus allowing multiple series of transductions.

Other vectors include plasmid vector, cosmid vector, bacterial artificial chromosome (BAC) vector, transposon-based vector. Plasmids may be delivered by a variety of parenteral, mucosal and topical routes. For example, the DNA plasmid can be injected by intramuscular, eye, intradermal, subcutaneous, or other routes. It may also be administered by intranasal sprays or drops, rectal suppository and orally. It may also be administered into the epidermis or a mucosal surface using a gene-gun. The plasmids may be given in an aqueous solution, dried onto gold particles or in association with another DNA delivery system including but not limited to liposomes, dendrimers, cochleate and microencapsulation.

In a preferred embodiment, the antisense oligonucleotide, siRNA, shRNA or related nucleic acid sequence is under the control of a heterologous regulatory region, e.g., a heterologous promoter. The promoter can also be, e.g., a viral promoter, such as CMV promoter or any synthetic promoters.

siRNA can also be directly conjugated with a molecular entity designed to help targeted delivery. Examples of conjugates are lipophilic conjugates such as cholesterol, or aptamer-based conjugates. Cationic peptides and proteins are also used to form complexes with a negatively charged phosphate backbone of the siRNA.

In another embodiment, such inhibitor of PI3K-AKT-mTOR pathway is a small molecule. Such inhibitors are well known in the art (see for instance Yap T A, Garrett M D, Walton M I, Raynaud F, de Bono J S, Workman P (2008). “Targeting the PI3K-AKT-mTOR pathway: progress, pitfalls, and promises”. Current Opinion in Pharmacology 8 (4): 393-412 (the content of which is incorporated herein by reference). Non-limiting examples of inhibitors of PI3K-AKT-mTOR pathway includes PI3K inhibitors, AKT inhibitors and mTOR inhibitors as described in detail below. It should be further noted that some compounds may inhibit several targets in PI3K-AKT-mTOR pathway. Thus, some compounds such as SF116 or BEZ235 are mTOR/PI3K dual inhibitors.

### PI3K Inhibitors

In one particular embodiment, the PI3K-AKT-mTOR pathway inhibitor is a PI3K inhibitor.

As used herein, the term “PI3K inhibitor” refers to a compound (natural or synthetic) which is effective to inhibit PI3K activity. In addition, the inhibitors with a specific activity on PI3K may be preferred. Inhibitors of PI3K are, in most cases, compounds that interfere with the binding of ATP in the binding site of PI3K ATP, thus preventing a more or less specific activity of these kinases. In some cases, inhibitors of PI3K are allosteric inhibitors.

Non-limiting examples of PI3K inhibitors include: NVP-BEZ235 (BEZ235) (Novartis); LY294002 (Cell Signaling #9901); GDC-0941 (Genentech/Roche); GDC-0980 (Genentech); PI-103 (Piramed); XL147 (Exilixis/Sanofi-Aventis); XL418 (Exilixis); XL665 (Exelixis); LY29002 (Eli Lilly); ZSTK474 (Zenyaku Kogyo); BGT226 (Novartis); wortmannin; quercetin; tetrodotoxin citrate (Wex Pharmaceuticals); thioperamide maleate; IC87114; PIK93; TGX-115; deguelin; NU 7026; OSU03012; tandutinib (Millennium Pharmaceuticals); MK-2206 (Merck); OSU-03012; triciribine (M.D. Anderson Cancer Center); PIK75; TGX-221; NU 7441; PI 828; WHI-P 154; AS-604850; AS-041164 (Merck Serono); AS-252424; AS-605240; AS-604850; compound 15e;17-P-hydroxywortmannin; PP121; WAY-266176; WAY-266175; BKM120 (Novartis); PKI-587 (Pfizer); BYL719 (Novartis); XL765 (Sanofi-Aventis); GSK1059615 or GSK615 (GlaxoSmithKline); IC486068; SF1126 (Semafore Pharmaceuticals); CAL-101 (Gilead Sciences); LME00084; PX-478 (Oncothyreon); PX-866 (Oncothyreon); PX-867 (Oncothyreon), BAY 80-6946 (Bayer), GSK2126458 (GlaxoSmithKline), INK1117 (Intellikine), IPI-145 (Infinity Pharmaceuticals) Palomid 529 (Paloma Pharmaceuticals); ZSTK474 (Zenyaku Kogyo); PWT33597 (Pathway Therapeutics); TG100-115 (TargeGen); CAL263 (Gilead Sciences); SAR245408 (Sanofi-Aventis); SAR245409 (Sanofi-Aventis); GNE-477; CUDC-907; and BMK120 (Novartis).

Exemplary PI3K inhibitors that are contemplated by the invention include but are not limited to, for example, those as described in the following international patent applications which are hereby incorporated by reference in their entireties: WO2008/027584, WO2008070150, 2,3-dihydroimidazo[1,2-c]quinazolines (WO2008/125833), 2-morpholin-4-yl-pyrimidines (WO2008/125835), pyrimidines (WO2008/125839), bicyclic heteroaryls (WO2009/010530), thiazolidinones (WO2009/026345), pyrrolothiazoles (WO2009/071888), tricyclic thiazole and thiophene derivatives (WO2009/071890), fused bicyclic thiazole and thiophene derivatives (WO2009/071895) and oxazole substituted indazoles (WO2010/125082).

Additional PI3K inhibitors are described in U.S. Pat. Nos. 6,100,090; 6,908,932; 7,598,377; and 7,666,901 (each herein incorporated by reference); and U.S. Patent Application Publication Nos. 2010/0069629; 2010/0034786; 2010/0029693; 2010/0022534; 2010/0016306; 2009/0325954; 2009/0318411; 2009/0247567; 2009/0233926; 2009/0227587; 2009/0118336; 2008/0319021; 2008/0269210; 2008/0242665; 2008/0085997; 2008/0039459; 2008/0132502; 2008/0014598; 2008/0287469; 2007/0244312; 2007/0238745; 2006/0089320; 2006/0026702; 2006/0084697; 2005/0272682; 2004/0077580; 2004/0063657; 2003/0182669; 2003/0158212; 2003/0149074; 2003/0225013; and 2003/0055018 (each herein incorporated by reference).

In one embodiment, the PI3K inhibitor is LY294002 (a morpholine derivative of quercetin) or 2-(4-Morpholinyl)-8-phenyl-4H-1-benzopyran-4-one. LY294002 may be obtained commercially or synthesized as described in U.S. Pat. No. 5,703,075, the content of which is incorporated herein by reference. In another embodiment, the PI3K inhibitor is a prodrug of LY294002 comprising a reversibly quaternized nitrogen as described in international patent application WO2004/089925. On example of such prodrug is SF1226 (Semafore Pharmaceuticals) which is composed of the PI3K inhibitor LY294002 conjugated to an RGD targeting peptide.

In a preferred embodiment, the PI3K inhibitor is selected from the group consisting of LY2940002, SF1126, PI103, GDC 0941, XL765, XL147, BGT226 and BEZ235.

### AKT Inhibitors

In one particular embodiment, the PI3K-AKT-mTOR pathway inhibitor is an AKT inhibitor.

As used herein, the term “AKT inhibitor” refers to a compound (natural or synthetic) that inhibits the signaling pathway AKT kinase (also called protein kinase B or PKB). Several chemical classes of small-molecule AKT inhibitors with varying potencies and specificities for the different AKT isoforms have now been developed. These include phosphatidylinositol analogs, ATP-competitive small molecules, pseudosubstrate compounds, and allosteric inhibitors.

Exemplary AKT inhibitors that are contemplated by the invention include but are not limited to, for example, those as described in the following international patent applications which are hereby incorporated by reference in their entireties: aminofurazans (WO2005/019190), substituted pyrimidines (WO2008/006040), and substituted pyridines (WO2009/032653).

In a preferred embodiment, the AKT inhibitor is selected from the group consisting of Perifosine, XL418, GSK690693, AT13148 and A-443654.

### mTOR Inhibitors

In one particular embodiment, the PI3K-AKT-mTOR pathway inhibitor is a mTOR inhibitor.

As used herein, the term “mTOR inhibitor” refers to a compound (natural or synthetic) that inhibits at least one activity of an mTOR, such as the serine/threonine protein kinase activity on at least one of its substrates (e.g., p70 S6 kinase 1, 4E-BP1, AKT/PKB and eEF2). A person skilled in the art can readily determine whether a compound, such as rapamycin or an analogue or derivative thereof, is an mTOR inhibitor. A specific method of identifying such compounds is disclosed in U.S. Patent Application Publication No. 2003/0008923.

In one embodiment, the mTOR inhibitor inhibits at least one activity of mTORC1. In another embodiment, the mTOR inhibitor inhibits at least one activity of mTORC2. In still another embodiment, the mTOR inhibitor inhibits at least one activity of mTORC1 and at least one activity of mTORC2. In one embodiment, the mTOR inhibitor is a compound that inhibits cell replication by blocking progression of the cell cycle from G1 to S by inhibiting the phosphorylation of serine 389 or threonine 412 of p70 S6 kinase.

In a preferred embodiment, the mTOR inhibitor is selected from the group consisting of rapamycin (also called sirolimus and described in U.S. Pat. No. 3,929,992), temsirolimus, deforolimus, everolimus, tacrolimus and rapamycin analogue or derivative thereof.

As used herein, the term “rapamycin analogue or derivative thereof” includes compounds having the rapamycin core structure as defined in U.S. Patent Application Publication No. 2003/0008923 (which is herein incorporated by reference), which may be chemically or biologically modified while still retaining mTOR inhibiting properties. Such derivatives include esters, ethers, oximes, hydrazones, and hydroxylamines of rapamycin, as well as compounds in which functional groups on the rapamycin core structure have been modified, for example, by reduction or oxidation. Pharmaceutically acceptable salts of such compounds are also considered to be rapamycin derivatives. Specific examples of esters and ethers of rapamycin are esters and ethers of the hydroxyl groups at the 42- and/or 31-positions of the rapamycin nucleus, and esters and ethers of a hydroxyl group at the 27-position (following chemical reduction of the 27-ketone). Specific examples of oximes, hydrazones, and hydroxylamines are of a ketone at the 42-position (following oxidation of the 42-hydroxyl group) and of 27-ketone of the rapamycin nucleus.

Examples of 42- and/or 31-esters and ethers of rapamycin are disclosed in the following patents, which are hereby incorporated by reference in their entireties: alkyl esters (U.S. Pat. No. 4,316,885); aminoalkyl esters (U.S. Pat. No. 4,650,803); fluorinated esters (U.S. Pat. No. 5,100,883); amide esters (U.S. Pat. No. 5,118,677); carbamate esters (U.S. Pat. No. 5,118,678); silyl ethers (U.S. Pat. No. 5,120,842); aminoesters (U.S. Pat. No. 5,130,307); acetals (U.S. Pat. No. 551,413); aminodiesters (U.S. Pat. No. 5,162,333); sulfonate and sulfate esters (U.S. Pat. No. 5,177,203); esters (U.S. Pat. No. 5,221,670); alkoxyesters (U.S. Pat. No. 5,233,036); O-aryl, -alkyl, -alkenyl, and -alkynyl ethers (U.S. Pat. No. 5,258,389); carbonate esters (U.S. Pat. No. 5,260,300); arylcarbonyl and alkoxycarbonyl carbamates (U.S. Pat. No. 5,262,423); carbamates (U.S. Pat. No. 5,302,584); hydroxyesters (U.S. Pat. No. 5,362,718); hindered esters (U.S. Pat. No. 5,385,908); heterocyclic esters (U.S. Pat. No. 5,385,909); gem-disubstituted esters (U.S. Pat. No. 5,385,910); amino alkanoic esters (U.S. Pat. No. 5,389,639); phosphorylcarbamate esters (U.S. Pat. No. 5,391,730); carbamate esters (U.S. Pat. No. 5,411,967); carbamate esters (U.S. Pat. No. 5,434,260); amidino carbamate esters (U.S. Pat. No. 5,463,048); carbamate esters (U.S. Pat. No. 5,480,988); carbamate esters (U.S. Pat. No. 5,480,989); carbamate esters (U.S. Pat. No. 5,489,680); hindered N-oxide esters (U.S. Pat. No. 5,491,231); biotin esters (U.S. Pat. No. 5,504,091); O-alkyl ethers (U.S. Pat. No. 5,665,772); and PEG esters of rapamycin (U.S. Pat. No. 5,780,462).

Examples of 27-esters and ethers of rapamycin are disclosed in U.S. Pat. No. 5,256,790, which is hereby incorporated by reference in its entirety.

Examples of oximes, hydrazones, and hydroxylamines of rapamycin are disclosed in U.S. Pat. Nos. 5,373,014, 5,378,836, 5,023,264, and 5,563,145, which are hereby incorporated by reference. The preparation of these oximes, hydrazones, and hydroxylamines is disclosed in the above listed patents. The preparation of 42-oxorapamycin is disclosed in U.S. Pat. No. 5,023,263, which is hereby incorporated by reference.

Other compounds within the scope of “rapamycin analog or derivative thereof” include those compounds and classes of compounds referred to as “rapalogs” in, for example, WO 98/02441 and references cited therein, and “epirapalogs” in, for example, WO 01/14387 and references cited therein.

Another compound within the scope of “rapamycin derivatives” is everolimus, a 4-O-(2-hydroxyethyl)-rapamycin derived from a macrolide antibiotic produced by Streptomyces hygroscopicus (Novartis). Everolimus is also known as Certican, RAD-001 and SDZ-RAD. Another preferred mTOR inhibitor is zotarolimus, an antiproliferative agent (Abbott Laboratories). Zotarolimus is believed to inhibit smooth muscle cell proliferation with a cytostatic effect resulting from the inhibition of mTOR. Another preferred mTOR inhibitor is tacrolimus, a macrolide lactone immunosuppressant isolated from the soil fungus Streptomyces tsukubaensis. Tacrolimus is also known as FK 506, FR 900506, Fujimycin, L 679934, Tsukubaenolide, PROTOPIC and PROGRAF. Other preferred mTOR inhibitors include AP-23675, AP-23573, and AP-23841 (Ariad Pharmaceuticals).

Preferred rapamycin derivatives include everolimus, CCI-779 (rapamycin 42-ester with 3-hydroxy-2-(hydroxymethyl)-2-methylpropionic acid; U.S. Pat. No. 5,362,718); 7-epi-rapamycin; 7-thiomethyl-rapamycin; 7-epi-trimethoxyphenyl-rapamycin; 7-epi-thiomethyl-rapamycin; 7-demethoxy-rapamycin; 32-demethoxy-rapamycin; 2-desmethyl-rapamycin; and 42-O-(2-hydroxyl)ethyl rapamycin (U.S. Pat. No. 5,665,772).

Additional mTOR inhibitors include TORC1 and TORC2 inhibitors. For example, OSI-027 (OSI Pharmaceuticals) is a small molecule TORC1/TORC2 inhibitor. OSI-027 inhibits both the TORC1 and TORC2 signaling complexes, allowing for the potential for complete truncation of aberrant cell signaling through this pathway.

In addition, torkinibs, ATP-competitive mTOR kinase domain inhibitors and inhibitors of both mTORC1 and mTORC2 may also be used according to the ionvention. Exemplary torkinibs include PP242 and PP30 (see, Feldman et al. (2009) PLoS Biology 7:371) and Torinl (Thoreen et al. (2009) J Biol Chem 284:8023).

In another aspect, the present invention provides a method of preventing or treating antiphospholipid syndrome (APS) in a patient comprising administering to the patient a therapeutically effective amount of a PI3K-AKT-mTOR pathway inhibitor.

In a further aspect, the present invention also provides a method of inhibiting or preventing APS-related vascular lesions in a patient comprising administering to the patient a therapeutically effective amount of a PI3K-AKT-mTOR pathway inhibitor.

In a still further aspect, the present invention provides a method of inhibiting endothelial mTORC activation triggered by APA in a patient in need thereof comprising administering to the patient a therapeutically effective amount of a PI3K-AKT-mTOR pathway inhibitor.

In a particular embodiment, the PI3K-AKT-mTOR pathway inhibitor is rapamycin (sirolimus).

In one embodiment, the patient may have developed or be at risk for developing APS. In one embodiment, the patient is a patient with antiphospholipid antibodies (APA).

By a “therapeutically effective amount” of a PI3K-AKT-mTOR pathway inhibitor as above described is meant a sufficient amount of the inhibitor to prevent or treat APS. It will be understood, however, that the total daily usage of the compounds and compositions of the present invention will be decided by the attending physician within the scope of sound medical judgment. The specific therapeutically effective dose level for any particular subject will depend upon a variety of factors including the disorder being treated and the severity of the disorder; activity of the specific compound employed; the specific composition employed, the age, body weight, general health, sex and diet of the subject; the time of administration, route of administration, and rate of excretion of the specific compound employed; the duration of the treatment; drugs used in combination or coincidential with the specific polypeptide employed; and like factors well known in the medical arts. For example, it is well within the skill of the art to start doses of the compound at levels lower than those required to achieve the desired therapeutic effect and to gradually increase the dosage until the desired effect is achieved. However, the daily dosage of the products may be varied over a wide range from 0.01 to 1,000 mg per adult per day. Preferably, the compositions contain 0.01, 0.05, 0.1, 0.5, 1.0, 2.5, 5.0, 10.0, 15.0, 25.0, 50.0, 100, 250 and 500 mg of the active ingredient for the symptomatic adjustment of the dosage to the subject to be treated. A medicament typically contains from about 0.01 mg to about 500 mg of the active ingredient, preferably from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 7 mg/kg of body weight per day.

The terms “treat”, “treating” or “treatment” refer to both therapeutic treatment and prophylactic or preventative measures, wherein the aim is to prevent or ameliorate APS or slow down (lessen) vascular lesions. Those in need of treatment include those already with the disorder as well as those in which the disorder is to be prevented.

The terms “preventing”, “prevention”, “preventative” or “prophylactic” refer to keeping from occurring, or to hinder, defend from, or protect from the occurrence of a condition, disease, disorder, or phenotype, including an abnormality or symptom. A patient in need of prevention may be prone to develop the condition.

### Pharmaceutical Compositions of the Invention

The PI3K-AKT-mTOR pathway inhibitor as described above may be combined with pharmaceutically acceptable excipients, and optionally sustained-release matrices, such as biodegradable polymers, to form therapeutic compositions.

Accordingly, the present invention also relates to a pharmaceutical composition for use in the prevention or treatment of APS comprising a PI3K-AKT-mTOR pathway inhibitor according to the invention and a pharmaceutically acceptable carrier.

The present invention also relates to a pharmaceutical composition for use in the prevention of APS-related vascular lesions comprising a PI3K-AKT-mTOR pathway inhibitor according to the invention and a pharmaceutically acceptable carrier.

In one embodiment, the pharmaceutical composition for use according to the invention comprises at least two PI3K-AKT-mTOR pathway inhibitors ((a) a PI3K inhibitor and an AKT inhibitor; (b) a PI3K inhibitor and a mTOR inhibitor; (c) an AKT inhibitor and a mTOR inhibitor; and (d) a PI3K inhibitor, an AKT inhibitor and a mTOR inhibitor as defined above).

In a particular embodiment, the PI3K-AKT-mTOR pathway inhibitor is rapamycin (sirolimus).

In one embodiment, the pharmaceutical composition for use according to the invention further comprises an additional therapeutic agent. In one particular embodiment, said additional therapeutic agent is an anti-thrombotic agent.

In one particular embodiment, the anti-thrombotic agent is heparin (unfractionated heparin or low molecular weight heparin or warfarin (or other vitamin K antagonists).

The present invention further relates to a pharmaceutical composition or a kit as defined below comprising a PI3K-AKT-mTOR pathway inhibitor according to the invention, an anti-thrombotic agent and a pharmaceutically acceptable carrier.

“Pharmaceutically” or “pharmaceutically acceptable” refers to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type.

In therapeutic applications, compositions are administered to a patient already suffering from a disease, as described, in an amount sufficient to cure or at least partially stop the symptoms of the disease and its complications. An appropriate dosage of the pharmaceutical composition is readily determined according to any one of several well-established protocols. For example, animal studies (for example on mice or rats) are commonly used to determine the maximal tolerable dose of the bioactive agent per kilogram of weight. In general, at least one of the animal species tested is mammalian. The results from the animal studies can be extrapolated to determine doses for use in other species, such as humans for example. What constitutes an effective dose also depends on the nature and severity of the disease or condition, and on the general state of the patient's health.

In prophylactic applications, compositions containing, for example PI3K-AKT-mTOR pathway inhibitors, are administered to a patient susceptible to or otherwise at risk of APS. Such an amount is defined to be a “prophylactically effective” amount or dose. In this use, the precise amount depends on the patient's state of health and weight.

In both therapeutic and prophylactic treatments, the inhibitor contained in the pharmaceutical composition can be administered in several dosages or as a single dose until a desired response has been achieved. The treatment is typically monitored and repeated dosages can be administered as necessary. Compounds of the invention may be administered according to dosage regimens established whenever inactivation of the PI3K-AKT-mTOR pathway is required.

The daily dosage of the products may be varied over a wide range from 0.01 to 1,000 mg per adult per day. Preferably, the compositions contain 0.01, 0.05, 0.1, 0.5, 1.0, 2.5, 5.0, 10.0, 15.0, 25.0, 50.0, 100, 250 and 500 mg of the active ingredient for the symptomatic adjustment of the dosage to the patient to be treated. A medicament typically contains from about 0.01 mg to about 500 mg of the active ingredient, preferably from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 10 mg/kg of body weight per day. It will be understood, however, that the specific dose level and frequency of dosage for any particular patient may be varied and will depend upon a variety of factors including the activity of the specific compound employed, the metabolic stability, and length of action of that compound, the age, the body weight, general health, sex, diet, mode and time of administration, rate of excretion, drug combination, the severity of the particular condition, and the host undergoing therapy.

In the pharmaceutical compositions of the present invention for oral, sublingual, subcutaneous, intramuscular, intravenous, transdermal, local or rectal administration, the active principle, alone or in combination with another active principle, can be administered in a unit administration form, as a mixture with conventional pharmaceutical supports, to animals and human beings. Suitable unit administration forms comprise oral-route forms such as tablets, gel capsules, powders, granules and oral suspensions or solutions, sublingual and buccal administration forms, aerosols, implants, subcutaneous, transdermal, topical, intraperitoneal, intramuscular, intravenous, subdermal, transdermal, intrathecal and intranasal administration forms and rectal administration forms.

### Kits of the Invention

In another aspect, the present invention also relates to a kit comprising at least two PI3K-AKT-mTOR pathway inhibitors of the invention, as a combined preparation for simultaneous, separate or sequential use in the prevention or the treatment of APS.

In still another aspect, the present invention further relates to a kit comprising at least two PI3K-AKT-mTOR pathway inhibitors of the invention, as a combined preparation for simultaneous, separate or sequential use in the prevention of APS-related vascular lesions.

In one embodiment, said at least two PI3K-AKT-mTOR pathway inhibitors are (a) a PI3K inhibitor and an AKT inhibitor; (b) a PI3K inhibitor and a mTOR inhibitor; (c) an AKT inhibitor and a mTOR inhibitor; and (d) a PI3K inhibitor, an AKT inhibitor and a mTOR inhibitor as defined above.

The terms “kit”, “product” or “combined preparation”, as used herein, define especially a “kit of parts” in the sense that the combination partners as defined above can be dosed independently or by use of different fixed combinations with distinguished amounts of the combination partners, i.e. simultaneously or at different time points. The parts of the kit of parts can then, e.g., be administered simultaneously or chronologically staggered, that is at different time points and with equal or different time intervals for any part of the kit of parts. The ratio of the total amounts of the combination partners to be administered in the combined preparation can be varied. The combination partners can be administered by the same route or by different routes. When the administration is sequential, the first partner may be for instance administered 1, 2, 3, 4, 5, 6, 12, 18 or 24 h before the second partner.

In one embodiment, the kit for use according to the invention further comprises an additional therapeutic agent. In one particular embodiment, said additional therapeutic agent is an anti-thrombotic agent.

The invention will be further illustrated by the following figures and examples. However, these examples and figures should not be interpreted in any way as limiting the scope of the present invention.

### FIGURES

FIG. 1: mTORC pathway is activated in kidney endothelial cells of patients with APS. Quantification of positive vascular section for P-AKT (Ser⁴⁷³), P-S6RP and PCNA. Scale bar: 50 μm. Data are means±SEM. Mann-Whitney test; APS/SLE APS+ versus Control/SLE APS−: *** P<0.001.

FIG. 2: APA activate mTORC pathway in endothelial cells. (A) Western blot and quantification of P-AKT (Ser⁴⁷³), P-S6RP and P-AKT (Thr³⁰⁸) in human micro vascular endothelial cells (HMEC) five minutes after exposition to NH IgG or APA IgG. (B) Effect of different inhibitors on mTORC1 and mTORC2 pathway. Western blot of P-AKT (Ser⁴⁷³) and P-S6RP in HMEC five minutes after exposition to NH IgG or APA IgG after exposure to PP242, LY294002, a short or a long exposure to sirolimus. Data are means±SEM. Mann-Whitney test; ** P<0.01; *** P<0.001. n=12-14 for in vitro experiments.

FIG. 3: Sirolimus prevents vascular lesions in transplant recipients with APA at 12-months post transplantation. (A) Renal vascular morphology of transplant recipients without antiphospholipid antibody (Tx APA−) and transplant recipients with antiphospholipid antibodies (Tx APA+) without (Siro−) or with sirolimus (Siro+). Percentage of biopsy with fibrous intimal hyperplasia lesion. (B) Banff scoring of kidney lesions from transplant recipients without antiphospholipid antibody (Tx APA−), transplant recipients with antiphospholipid antibodies without sirolimus (Tx APA+ Siro−) and with sirolimus (APA+ Siro+). (C) Measured glomerular filtration rate (mGFR) at 12 months post transplantation in the three groups of transplant recipients, APA−, APA+ Siro− and APA+ Siro+. (D) Allograft survival rate between the three groups of patients APA−, APA+ Siro− and APA+ Siro+. Data are means±SEM. ANOVA followed by Tukey-Kramer test; Tx APA+ Siro− versus Tx APA−: ### P<0.001; Tx APA+ Siro− versus Tx APA+ Siro+: *** P<0.001, Tx APA+ Siro+ versus Tx APA−: ^{◯◯}P<0.01.

FIG. 4: Sirolimus inhibits endothelial mTORC pathway activation. (A) Quantification of the number of vessels that co-expressed CD105 (endothelial cell marker) and P-PAKT (Ser⁴⁷³) per biopsy of transplant recipients without patients antiphospholipid antibody (Tx APA−), transplant recipients with antiphospholipid antibodies without (Tx APA+ Siro−) or with sirolimus (Tx APA+ Siro+). (B) Quantification of the number of vessels that co-expressed α-SMA and P-S6RP per biopsy of transplant recipients without patients antiphospholipid antibody (Tx APA−), transplant recipients with antiphospholipid antibodies without (Tx APA+ Siro−) or with sirolimus (Tx APA+ Siro+). (C) Quantification of PCNA-positive vascular section in biopsies of transplant recipients without patients antiphospholipid antibody (Tx APA−), transplant recipients with antiphospholipid antibodies without (Tx APA+ Siro−) or with sirolimus (Tx APA+ Siro+). Quantification of the number of vessels with at least one positive cell for PCNA per biopsy. Scale bar: 50 μm. Data are means±SEM. ANOVA followed by Tukey-Kramer test; Tx APA+ Siro− versus Tx APA−: ### P<0.001; Tx APA+ Siro− versus Tx APA+ Siro+: *** P<0.001.

### EXAMPLE

### Material & Methods

Patients and Data Collection:

Native Kidney Diseases:

To analyse mTORC activation in APSN on native kidneys, the inventors studied four distinct groups of patients followed in the Nephrology Department of Necker Hospital (Table 1). Briefly, they examined (i) a group of patients with primary APS associated with biopsy proven primary APSN (n=12), (ii) a group of patients with APSN due to secondary APS superimposed on SLE nephritis (class III or IV) (SLE APS+, n=20), (iii) a group of patients with SLE nephritis (class III or IV) but without APS nor APSN (SLE APS−, n=25), (iv) and a control group composed of healthy renal peritumoral tissues from patients who undergone partial or complete nephrectomy for renal neoplasia (controls, n=10). For each patient, renal function was determined using the MDRD formula at the time of biopsy.

TABLE 1
Demographic and clinical characteristics of patients with native
kidney disease:

| | | Primary | SLE | |
|---|---|---|---|---|
| | Controls | APS | APS− | SLE APS+ |
| Characteristics | (n = 10) | (n = 12) | (n = 25) | (n = 20) |
| Age at biopsy (years) | 48 ± 13 | 43 ± 24 | 28 ± 12 | 27 ± 16 |
| Female (%) | 33 | 75 | 68 | 70 |
| eGFR at the time of biopsy | 88 ± 12 | 46 ± 5 | 47 ± 15 | 49 ± 23 |
| (mL/min/1.73 m²) | | | | |
| Lupus anticoagulant (%) | 0 | 100 | 0 | 80 |
| Anti-β2 GP1 antibodies (%) | 0 | 83 | 0 | 85 |
| Anti-cardiolipin | 0 | 83 | 0 | 100 |
| antibodies (%) | | | | |

APS: Antiphospholipid Syndrome;
SLE APS−: Systemic Lupus Erythematosus without Antiphospholipid Syndrome;
SLE APS+: Systemic Lupus Erythematosus with Antiphospholipid Syndrome;
eGFR: estimated Glomerular Filtration Rate.
Data are means ± SEM.

Transplant Recipients:

The cohort of transplant recipients was previously described¹⁵, and the demographic characteristics are summarized in Table 2. Briefly, the inventors studied a first group of transplant recipients with APA+ (Tx APA+, n=37) and a control group of transplants recipients without APA− (Tx APA−, n=59) engrafted during the same period. These patients were followed in the Transplant unit of Necker Hospital. All patients with functioning allograft had surveillances biopsies and measured glomerular filtration rate (mGFR) at 3- and 12-months post transplantation. Patients had similar immunosuppressive regimen consisting in steroids, purine inhibitor and calcineurine inhibitor, except for thirteen and ten patients in the Tx APA− and Tx APA+ group, respectively, that received sirolimus starting at day 0 instead of calcineurin inhibitor.

TABLE 2
Demographic and clinical characteristics of kidney transplant recipients:

| | | Tx aPL+ (n = 37) | |
|---|---|---|---|
| | Tx aPL− | Sirolimus− | Sirolimus+ |
| Characteristics | (n = 59) | (n = 27) | (n = 10) |
| Age at transplantation (years) | 54 ± 11 | 51 ± 11 | 47 ± 14 |
| Female (%) | 40 | 50 | 40 |
| Age at time of ESRD (years) | 47 ± 12 | 40 ± 1 | 45 ± 22 |
| Duration of HD (months) | 55 ± 37 | 51 ± 27 | 56 ± 16 |
| Steroids (%) | 88 | 92 | 90 |
| Calcineurin inhibitors (%) | 78 | 100 | 0 |
| mTORC inhibitor (%) | 22 | 0 | 100 |
| MMF/MA/Aza (%) | 67/21/12 | 44/37/19 | 100/0/0 |
| 12-Mo Tacrolimus trough | 10.0 ± 6.0 | 10.3 ± 5.9 | NA |
| levels (ng/mL) | | | |
| 12-Mo Cyclosporine peak | 642 ± 344 | 643 ± 365 | NA |
| levels (ng/mL) | | | |
| 12-Mo Sirolimus levels | 16 ± 2 | NA | 18 ± 9 |
| (ng/mL) | | | |
| Lupus anticoagulant (%) | 0 | 100 | 100 |
| Anti-β2GPI antibodies (%) | 0 | 18 | 20 |
| Anti-cardiolipin antibodies | 0 | 26 | 40 |
| (%) | | | |

Tx aPL−: Transplant recipients without Antiphospholipid Antibodies;
Tx aPL+: Transplant recipients with Antiphospholipid Antibodies;
ESRD: End Stage Renal Disease;
HD: Hemodialysis;
MMF: Mycophenolate mofetil;
MA: Mycophenolic acid;
Aza: Azathioprine;
12-Mo: 12 months post-transplantation;
NA: Not Applicable.
Data are means ± SEM.

Autopsy Cases:

The inventors studied two groups of deceased patients autopsied in the Department of Pathology of La Pitié Salpétrière (Table 3). The first group of patients had developed a catastrophic antiphospholipid syndrome (CAPS) (n=4), whereas the second group only displayed SLE without APS (n=4).

Informed written consent was obtained from each patient.

TABLE 3
Demographic and clinical characteristics of autopsy cases:

| | | | | | Atherosclerosis |
|---|---|---|---|---|---|
| Group | Patient | Age | Gender | Cause of death | in large vessels |
| SLE APS− | 1 | 78 | Male | Retroperitoneal | Yes |
| | | | | hemorrhage | |
| | 2 | 49 | Female | Aortic | No |
| | | | | dissection | |
| | 3 | 52 | Female | Disseminated | Yes |
| | | | | tuberculosis | |
| | 4 | 37 | Female | Septic shock | Yes |
| SLE APS+ | 1 | 46 | Male | CAPS | No |
| | 2 | 36 | Female | CAPS | No |
| | 3 | 31 | Male | CAPS | No |
| | 4 | 50 | Female | CAPS | Yes |

SLE APS−: systemic lupus erythematosus without antiphospholipid syndrome;
SLE APS+: systemic lupus erythematosus with antiphospholipid syndrome;
CAPS: catastrophic antiphospholipid syndrome.

Renal Function, Cyclosporine, Tacrolimus and Sirolimus Serum Levels:

The serum creatinine level was measured using a Synchron Cx4 autoanalyzer (Beckman Coulter, Villepinte, France). The glomerular filtration rate (GFR) was evaluated by iohexol clearance at 3- and 12-months post transplant as previously described²⁵. Cyclosporine and tacrolimus serum levels were determined by radioimmunoas say and sirolimus serum levels by high-performance liquid chromatography²⁶.

Biopsy samples and morphological analysis: Human kidney biopsies were either fixed, in alcoholic Bouin's solution (native kidneys) or in alcohol-formalin-acetic acid solution (transplant kidneys). Carotid and left anterior descending arteries from autopsy cases were fixed in formalin. All samples were then embedded in paraffin. Four-μm sections were stained with Periodic Acid Schiff (PAS), Masson's trichrome and hematoxyin and eosin (H&E). Schiff (PAS), Masson's trichrome and hematoxylin and eosin (HE). Kidney biopsies were independently examined and scored by two pathologists at Necker Hospital. Lesions related to APS-nephropathy were quantified according to the usual criteria¹². For each biopsy, the number of vessels displaying fibrous intimal hyperplasia were counted in all the fields of the section, and the data were expressed as the number of damaged vessels for the total number of vascular sections. Lesions related to allograft nephropathy were evaluated according to the Banff classification²⁷, which takes into account glomerular, tubular and interstitial changes. Autopsy cases were carefully examined by two pathologists at La Pitie Salpetiere Hospital. Vascular lesions, and in particular fibrous intimal hyperplasia, were searched for and characterized in all the tissues.

Functional Studies:

AKT and S6RP activation were evaluated using immunohistochemistry, immunofluorescence and co-localization experiments. The ability of aPL to modulate the mTORC pathway was studied in human microvascular endothelial cells incubated with IgG obtained either from patients with APS (n=12) or from healthy volunteers (n=14) (Table 4)

TABLE 4
Antiphospholipid antibody titers used in the in vitro study:

| | Anti-cardiolipin | Anti-β2-GPI | |
|---|---|---|---|
| Serum | (>10 GPL units) | (>12 GPL units) | Lupus Anticoagulant |
| 1 | 97 | 80 | + |
| 2 | 27 | 26 | + |
| 3 | 580 | 480 | + |
| 4 | 61 | 20 | + |
| 5 | 174 | 20 | + |
| 6 | 64 | 130 | + |
| 7 | 85 | 90 | + |
| 8 | 110 | 41 | + |
| 9 | 16 | 18 | + |
| 10 | 480 | 340 | + |
| 11 | 91 | 90 | + |
| 12 | 330 | 35 | + |

Immunohistochemistry and Immunofluorescence:

Immunofluorescence:

Four-μm sections of paraffin-embedded kidneys were incubated with P-AKT (Ser⁴⁷³) antibody (Cell Signaling Technology), anti-P-S6RP antibody (Cell Signaling Technology), anti-αSMA antibody (Sigma-Aldrich), anti-KI67 antibody (Novus Biological), anti-CD31 antibody (Dako) and anti-CD105 antibody (Sigma-Aldrich) after appropriate antigen retrieval. The primary antibodies were revealed with the appropriate Alexa 488- or 555-conjugated secondary antibody (Molecular Probes). Immunofluorescence staining was acquired using the Zeiss LSM 700 confocal microscope.

The inventors counted among all vascular section on a biopsy, the number of vessels that co-expressed either α-SMA and P-S6RP or CD105 and P-PAKT (Ser⁴⁷³). Thus, for each biopsy, the number of vessels that co-expressed either CD105 and P-PAKT (Ser⁴⁷³) or α-SMA and P-S6RP was determined in all the fields of the section, and the data were expressed as the number of positive vessels for the total number of vascular sections. They only focused on arteries and arterioles, as capillaries are not involved in APS vascular lesions¹².

Immunohistochemistry:

4-μm sections of paraffin-embedded kidneys were incubated with P-AKT (Ser⁴⁷³) antibody (Cell Signaling Technology), anti-P-S6RP antibody (Cell Signaling Technology) after the appropriate antigen retrieval. The primary antibodies were revealed with the appropriate secondary antibody (Molecular Probes). Peroxidase activity was revealed by 3-30-diamino-benzidine-tetrahydrochloride (DAB, Dako).

Cell Proliferation Assay:

Proliferative cells were detected in kidney using proliferating cell nuclear antigen (PCNA) immunostaining. 4-μm sections of paraffin-embedded kidneys were incubated with a mouse anti-PCNA antibody (DAKO) followed by a secondary mouse antibody (Molecular Probes). The staining was revealed by DAB. The inventors counted among all vascular section on a biopsy, all vessels with at least a PCNA positive cell. The vascular proliferation index was calculated as the number of vessels with at least one PCNA-positive nucleus out of the total number of vessels. In the second approach, paraffin-embedded sections were incubated with both anti-αSMA antibodies (Sigma-Aldrich) and anti-Ki67 antibodies (Novus Biological) after appropriate antigen retrieval. The primary antibodies were revealed with the appropriate Alexa 488- or 555-conjugated secondary antibodies (Molecular Probes). The vascular proliferation index was calculated as the number of α-SMA-positive vessels with at least one Ki67-positive nucleus out of the total number of vessels. All the microscopic fields of the section were quantified for each antibody.

IgG Purification:

Human IgG containing antiphospholipid antibodies (APA IgG) were obtained from 12 patients with APS, and control human IgG from volunteers IgG (n=14) were purified using the Melon Gel IgG Purification System (Thermo Scientific). The details of the antibodies used for the in vitro studies are summarized in Table 4, but in brief, aPL IgG was isolated from 7 patients with biopsy-proven APSN and 5 patients with either CAPS, preeclampsia or lethal pulmonary embolism. The IgG was purified using the Melon Gel IgG Purification System (Thermo Scientific). The purity of the IgG preparations was assessed by SDS-PAGE on a 7% acrylamide gel that was stained with Coomassie.

Lupus Anticoagulant (LA) was detected using a combination of different procedures, including the kaolin clotting time (KCT), the dilute Russell viper venom test (dRVVT), the APTT and the Rosner index. Anticardiolipin and anti-β2-GPI antibodies were measured with ELISA as previously described⁵⁸. For the anticardiolipin antibodies, values were expressed as GPL units (1 GPL unit=1 μg of affinity-purified IgG anticardiolipin from an original index serum sample) and considered positive when >10 GPL units were reported. Both IgG and IgM anticardiolipin antibodies were determined. For anti-β2-GPI antibodies, values were expressed as GPL units and considered positive when >12 GPL units were reported.

Cells Culture and Cells Experiments:

Human micro vascular endothelial cells (HMEC) were cultured in MCDB 131 medium supplemented with 10% FCS (MCDB 131-10% FCS medium). HMEC were grown to 80% of confluence then starved for 12 h in MCDB 131 medium supplemented with 2% FCS (MCDB 131-2% FCS medium). Cells were next incubated with β2-GP1 (5 μg/ml, Stago) in MCDB 131-2% FCS medium for 1 hour at 37° C. After washing, HMEC were exposed either to NH IgG (100 μg/mL) (n=14) or APA IgG (100 μg/mL) (n=12) antibodies in MCDB 131-2% FCS medium for 5 minutes.

HMEC were incubated during 1 hour (short exposure) or 48 hours (long exposure) with sirolimus (LC laboratories, L-7962) 20 nM, then exposed during 1 hour to β2-GPI with sirolimus 20 nM and finally exposed to NH IgG (n=6) or APA IgG (n=6) during five minutes. For LY294002 (20 μM) (LC laboratories, R-5000) and PP242 (0.5 μM) (Azasynth) similar experimental procedures were used but HMEC were pretreated before use during one hour. All experiments were performed in duplicates.

Western Blot:

Western blots were performed as previously described²⁸. Briefly, protein extracts from HMEC were resolved by SDS-PAGE before being transferred onto membrane and incubated with anti-P-AKT (Ser⁴⁷³) antibody (Cell Signaling Technology), anti-P-AKT (Thr³⁰⁸) antibody (Cell Signaling Technology), anti-P-S6RP antibody (Cell Signaling Technology), anti-AKT antibody (Cell Signaling Technology), anti-S6RP antibody (Cell Signaling Technology) and anti-βactin antibody (Sigma-Aldrich). Images were acquired using Fusion FX7 system (Vilber Loumart) and analysed using Bio-1D software (Vilber Loumart).

Data Analysis and Statistics:

Data were expressed as means±SEM. Differences between the experimental groups were evaluated using ANOVA, followed when significant (P<0.05) by the Tukey-Kramer test. When only two groups were compared, Mann-Whitney tests were used. The statistical analysis was performed using Graph Prism Software.

Results

1/ mTORC Pathway is Activated in Endothelial Cells of Patients with APA:

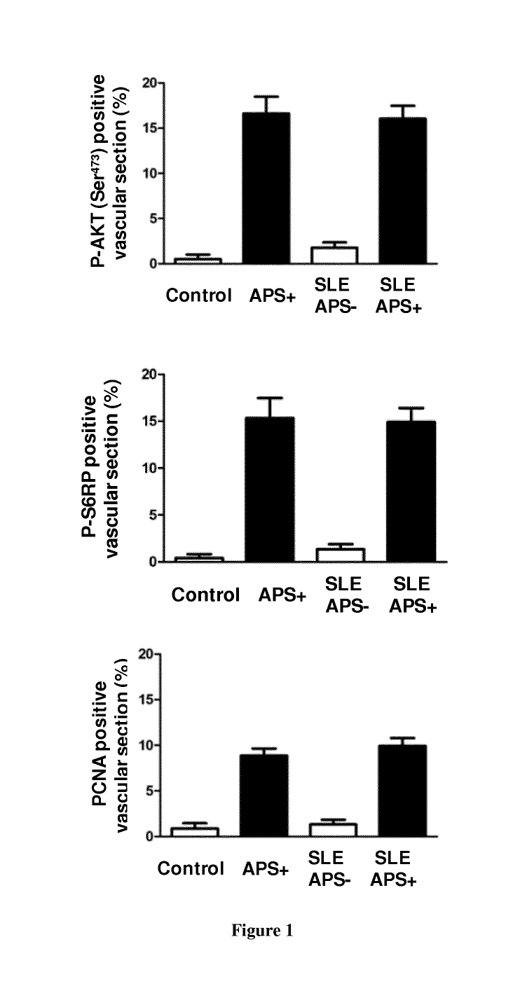

In order to investigate the state of activation of mTORC pathway in renal vessels of patients with primary APSN the inventors analysed the phosphorylation of S6RP and AKT (Ser⁴⁷³), which reflect the activation of mTORC1 and mTORC2, respectively. Immunostaining on serial section of renal vessels from patients with APSN showed that mTORC1 and mTORC2 were activated. To further characterize mTORC activation in vessels of patients with APSN we performed colocalization study with vessels markers. Immunofluorescence revealed a high kidney number P-S6RP and P-AKT (Ser⁴⁷³) positive vascular sections in kidney biopsies from patients with primary APS whereas any signal could be detected in controls (FIG. 1). Interestingly, most of the positive cells localized to vascular sections with prominent lesions. Colocalization experiments showed that both P-S6RP and P-AKT (Ser⁴⁷³) were activated in endothelial cells (CD105 positive cells) but not in α-SMA positive cells. In addition, immunostaining on serial section showed that mTORC1 and mTORC2 activation occurred in the same vessels.

The inventors then investigate if mTORC pathways were also activated in endothelial cells of patients with secondary APSN. They took advantage of a cohort of patients with SLE complicated by APS (SLE APS+) or not (SLE APS−). By comparing kidney biopsies from patients with similar degree of lupus nephritis, they observed that P-S6RP and P-AKT (Ser⁴⁷³) positive vascular sections were almost exclusively detected in the SLE APS+ group. Colocalization experiments confirmed the endothelial nature of mTORC activation (FIG. 1). Collectively these results indicate that the occurrence of renal vascular lesions is strongly and specifically associated with mTORC1 and mTORC2 activation in APS patients.

2/ Endothelial mTORC Pathway is Activated During CAPS:

To determine if APA induces vascular lesion is restricted to kidney or associated with a more general vascular disease, they explored autopsy cases with CAPS and SLE as a control group. Compared to SLE, they observed in two different vascular beds studied (carotid and left anterior descending artery), severe and extraordinary narrowed lumen of vessels by neointimal formation in both territories specifically in the CAPS group. They observed, on serial sections, that all narrowed vessel in the CAPS group had positives endothelial cells for P-S6RP and P-AKT (Ser⁴⁷³) whereas SLE APS− patients did not. Of note, they observed in this particular setting that few cells in the neointima were also positive for mTORC pathway activation. These cells displayed features of infiltrating inflammatory cells. Hence, APA induces systemic vascular lesions with endothelial mTORC pathway activation.

3/ mTORC Activation is Associated with Vascular Proliferation in APSN:

Since morphological appearance of the intima suggests hypercellularity and, as mTOR pathway activation is associated with proliferation, they hypothesized that vascular cell proliferation might contribute to the development of lesions. PCNA immunostaining showed that very few cells were positive in vessels of the control group and the SLE APL− group. Remarkably, the number of vascular cross section with PCNA positive cells dramatically increased in the group of patients with either primary APS or SLE APS+ (FIG. 1). Colocalization studies using antibodies directed against Ki-67 and α-SMA showed that proliferation was not restricted to endothelial cells but also involved VSMC, suggesting potential crosstalk between the two cellular compartments, as previously reported in other pathological contexts.

4/ APA Trigger mTORC Pathway Activation in Endothelial Cells:

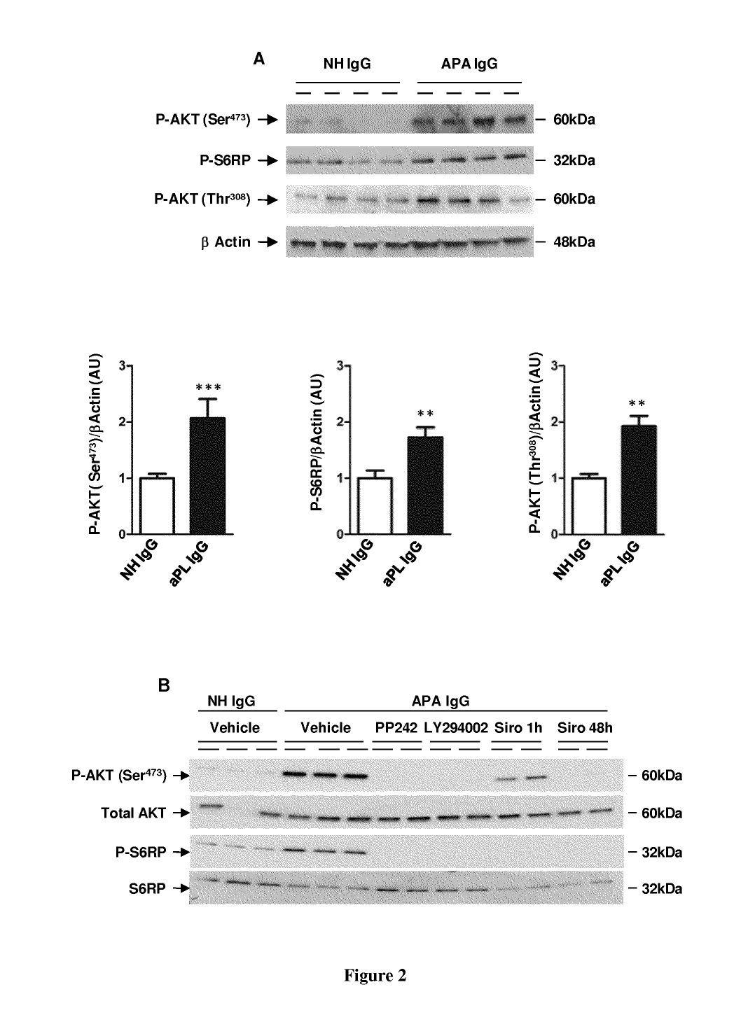

The inventors next investigate if APA may directly activate mTORC pathway in endothelial cells. In this aim, the inventors incubated a line of HMEC with either normal human IgG obtained from healthy individuals (NH IgG) or polyclonal APA isolated from APS patients (APA IgG). Strikingly, APA IgG induced a marked increase in the phosphorylation of S6RP and AKT (Ser⁴⁷³) within five minutes whereas any activation was observed in NH IgG did not (FIG. 2A). To characterize the mTORC implication in APA induces AKT activation, they then pretreated HMEC with PP242, a selective mTOR kinase inhibitor²⁹. They observed that this treatment completely abolished APA− induces AKT and S6RP phosphorylation.

Since AKT could be recruited to cell membrane by Phosphoinositide-Dependent Protein kinase 1 (PDK1)³⁰, they investigated the phosphorylation status of AKT on Thr³⁰⁸. Interestingly, we observed that APA IgG induced a marked increase in the phosphorylation of AKT (Thr³⁰⁸). More importantly, pretreatment of HMEC with LY294002, a PI3K inhibitor³¹, was able to completely prevent the activation of the AKT pathway supporting the role of a PI3K dependent recruitment of AKT to cell membrane (FIG. 2B).

Since sirolimus is a specific inhibitor of mTORC routinely used in clinics, we evaluate the effect of this drug on APA induced AKT activation (FIG. 2B). Sirolimus inhibits mTORC1 by dissociating the mTORC1 complexes, but also depending of cell type and treatment duration, sirolimus has been shown to inhibit mTORC2, likely by preventing the assembly of novel mTORC2 complexes³²⁻⁴¹. Consistent with these findings, the inventors observed that a short exposure of HMEC to sirolimus led to a complete inhibition of the APA− induces phosphorylation of S6RP but failed to prevent AKT phosphorylation on the residue Ser⁴⁷³. Interestingly, after a longer exposition, sirolimus blocked the APA− induces phosphorylation of both S6RP and AKT (FIG. 2B).

Collectively these results indicate that APA activates mTORC2 and mTORC1 in endothelial cells in a PI3K dependent manner.

5/ Sirolimus Inhibits Endothelial mTORC Pathway Activation and Prevents Vascular Lesions in Transplant Recipients with APA:

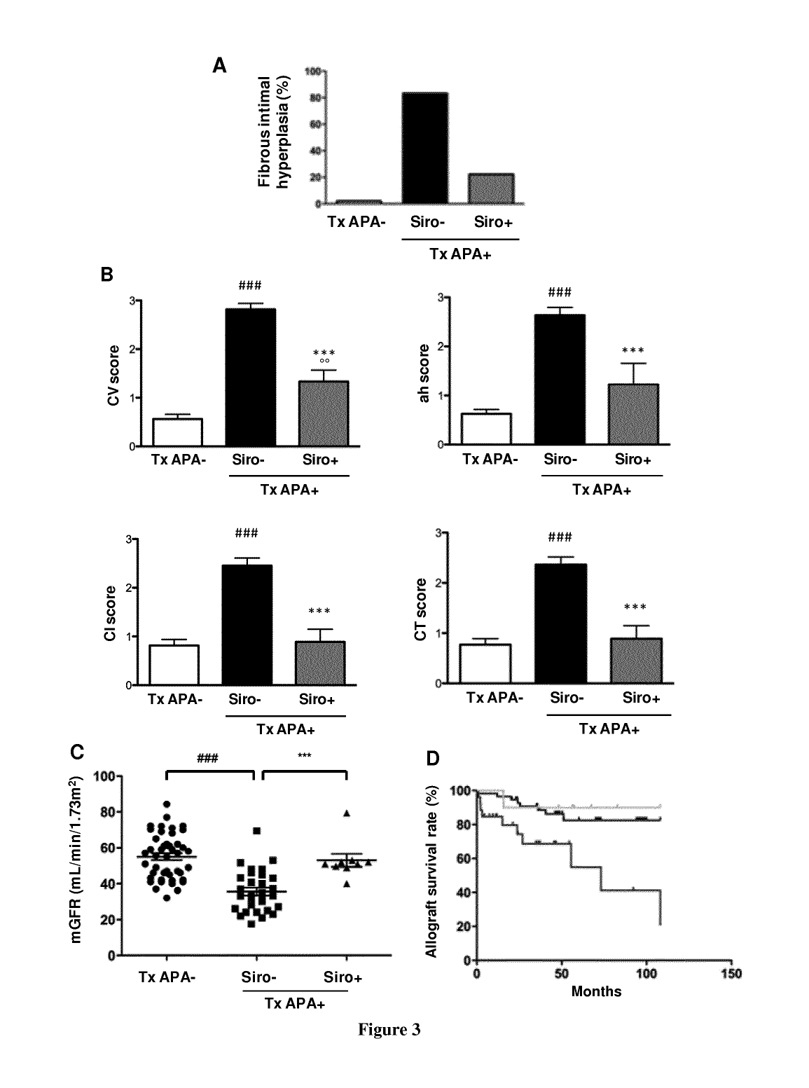

The present results suggested that sirolimus could be a potential therapeutic for APSN. The inventors took advantages of the use of this compound as an immunosuppressive drug in renal transplantation. They recently reported that patients with APA (Tx APA+) at the time of transplantation tend to develop severe vascular lesions on the grafted kidney resulting in a poor functional outcome¹⁵. Among the 37 Tx APA+ patients of our cohort, 10 received sirolimus therapy as an immunosuppressive regimen (Tx APA+ Siro+). Using protocol biopsies, they first observed that, while pre-implantation biopsies were similar in all groups, Tx APA+ Siro+ patients developed only very few chronic APSN lesions, such as intimal hyperplasia (FIG. 3A), and less non-specific chronic allograft lesions of the vessels, interstitium and tubules, during the first year of transplantation compared to transplant recipients with APA but without sirolimus (Tx APA+ Siro−) (FIG. 3B). Of note, Banff scoring of preimplantation biopsies was not different between patients in the Tx APA−, Tx APA+ Siro− and Tx APA+ Siro+. Moreover, Tx APA+ Siro+ patients had a significantly better measured glomerular filtration rate (mGFR) compared to APA+ Siro− patients (56±10.8 vs. 39.6±14.6 mL/min respectively) (FIG. 3C). Importantly, after a median follow-up of 52.5±23.5 months, Kaplan Meier survival analysis showed a significantly improved allograft survival rate in Tx APA+ Siro+ patients compared to Tx APA+ Siro− patients (FIG. 3D).

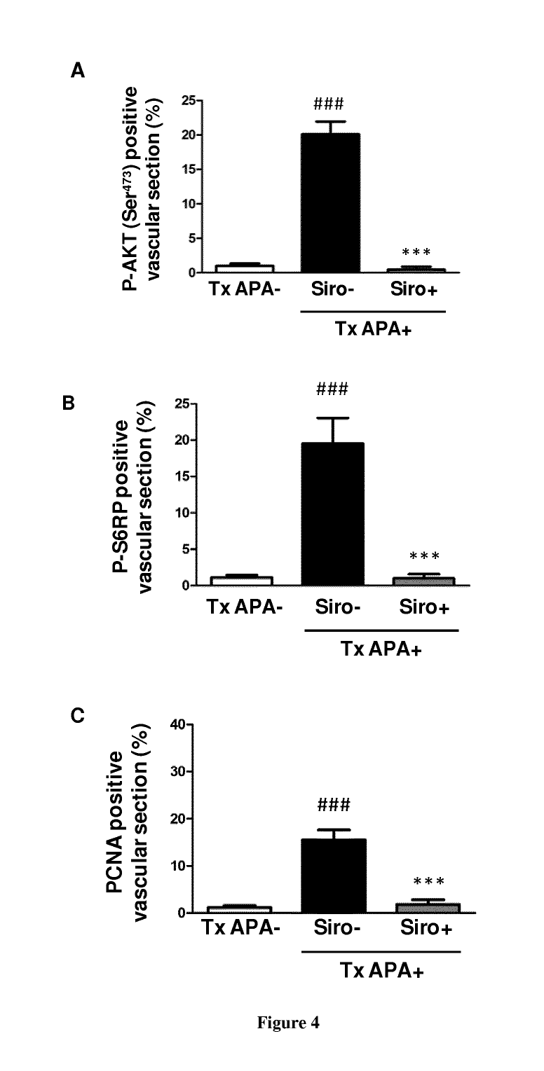

The inventors then evaluated using protocols biopsies, the state of endothelial mTORC activation. They observed that, either at 3- or 12-months post transplantation, a high number of P-AKT (Ser⁴⁷³) and P-S6RP positive renal vascular sections was present in biopsies from Tx APA+ Siro− patients whereas only very few vascular sections were positive in the Tx APA− group (FIGS. 4A and 4B). This observation corroborates the crucial role of APA to trigger mTOR pathway in endothelial cells. Importantly, we failed to detect any mTORC1 and also mTORC2 activity, in vascular section of Tx APA+ Siro+ patients (FIGS. 4A and 4B). Next, they analysed the rate of vascular cells proliferation on protocol biopsies to assess the impact of endothelial mTORC activation during the post transplant course. As on native kidneys, they observed that mTORC activation was associated with an increase of vascular cells proliferation in Tx APA+ Siro− patients compared to Tx APA− patients (FIG. 4C). Strikingly, vascular cell proliferation was dramatically reduced in Tx APA+ Siro+ patients (FIG. 4C).

A careful analysis of the other clinical variables known to affect graft outcome revealed that none of them accounted for the prolonged kidney survival in Tx aPL+ Siro+ patients. In particular, the immunological variables (microcirculation inflammation, C4d expression) were comparable in the three groups of patients (Table 5). Similarly, the titles and the types of aPL were similar in Tx aPL+ recipients, regardless of the immunosuppressive regime used (Table 6). Efficient anticoagulant medications were also comparable in these patients. Consistently, no thrombotic lesions were detected in the damaged vessels of Tx aPL+ recipients, regardless of sirolimus administration.

TABLE 5
Microcirculation inflammation in kidney biopsies of the three
groups of recipients according to the Banff classification:

| | Month 3 post-Tx | | | Month 12 post-Tx | | |
|---|---|---|---|---|---|---|
| Groups | g | ptc | C4d | 0 | ptc | C4d |
| Tx aPL− | 0.2 ± 0.1 | 0.2 ± 0.1 | 0.1 ± 0.3 | 0.2 ± 0.1 | 0.2 ± 0.1 | 0.1 ± 0.4 |
| Tx aPL+ Siro− | 0.3 ± 0.6 | 0.4 ± 0.9 | 0.1 ± 0.4 | 0.1 ± 0.5 | 0.1 ± 0.4 | 0.1 ± 0.5 |
| Tx aPL+ Siro+ | 0.1 ± 0.4 | 0.1 ± 0.4 | 0.2 ± 0.4 | 0.1 ± 0.3 | 0.1 ± 0.3 | 0.1 ± 0.3 |

Tx: Transplantation; g: Glomerulitis; ptc: Peritubular Capillaritis; C4d: C4 deposits; Tx aPL: Transplant recipients without Antiphospholipid Antibodies; Tx aPL+ Siro−: Transplant recipients with Antiphospholipid Antibodies without sirolimus; Tx aPL+ Siro+: Transplant recipients with Antiphospholipid Antibodies treated with sirolimus. Data are means ± SEM.

TABLE 6
Antiphospholipid antibody liters during the first year of transplantation:

| | | Day 0 | | | Month 12 post-Tx | | |
|---|---|---|---|---|---|---|---|
| | | | Anti-cardiolipin | Anti-β2GPI | | Anti-cardiolipin | Anti-β2GPI |
| | Patient | LA | (>10 GPL units) | (>12 GPL units) | LA | (>10 GPL units) | (>12 GPL units) |
| Tx aPL+ | 1 | + | 0 | 0 | NA | NA | NA |
| Siro− | 2 | + | 0 | 0 | + | 0 | 0 |
| | 3 | + | 0 | 0 | + | 0 | 0 |
| | 4 | + | 18 (IgM) | 0 | + | 16 (IgM) | 0 |
| | 5 | + | 0 | 0 | NA | NA | NA |
| | 6 | + | 0 | 287 (IgG) | NA | NA | NA |
| | 7 | + | 0 | 16 (IgM) | + | 0 | 41 (IgG) |
| | 8 | + | 0 | 226 (IgG) | NA | NA | NA |
| | 9 | + | 0 | 0 | + | 0 | 0 |
| | 10 | + | 0 | 0 | + | 0 | 0 |
| | 11 | + | 0 | 0 | + | 0 | 0 |
| | 12 | + | 0 | 0 | + | 0 | 0 |
| | 13 | + | 0 | 0 | NA | NA | NA |
| | 14 | + | 0 | 0 | + | 0 | 0 |
| | 15 | + | 0 | 0 | + | 0 | 0 |
| | 16 | + | 29 (IgG) | 0 | + | 41 (IgG) | 0 |
| | 17 | + | 0 | 0 | + | 0 | 0 |
| | 18 | + | 18 (IgM) | 0 | + | 38 (IgM) | 0 |
| | 19 | + | 36 (IgG); 15 (IgM) | 0 | + | 29 (IgG); 25 (IgM) | 0 |
| | 20 | + | 0 | 0 | + | 0 | 0 |
| | 21 | + | 47 (IgG) | 0 | + | 102 (IgG) | 0 |
| | 22 | + | 0 | 0 | NA | NA | NA |
| | 23 | + | 0 | 0 | + | 0 | 0 |
| | 24 | + | 0 | 11 (IgG) | + | 0 | 0 |
| | 25 | + | 93 (IgG) | 70 (IgG) | + | 145 (IgG) | 110 (IgG) |
| | 26 | + | 44 (IgG); 24 (IgM) | 0 | + | 27 (IgG) | 0 |
| | 27 | + | 0 | 0 | + | 0 | 0 |
| | Mean (%) | 100 | 26 | 19 | 100 | 33 | 10 |
| Tx aPL+ | 28 | + | 0 | 0 | + | 0 | 0 |
| | 29 | + | 0 | 25 (IgG) | + | 0 | 42 (IgG) |
| | 30 | + | 76 (IgG) | 0 | + | 55 (IgG) | 0 |
| | 31 | + | 29 (IgG); 32 (IgM) | 0 | + | 58 (IgG) | 0 |
| | 32 | + | 0 | 0 | + | 0 | 0 |
| | 33 | + | 0 | 0 | NA | NA | NA |
| | 34 | + | 19 (IgM) | 0 | + | 43 (IgG) | 0 |
| | 35 | + | 0 | 0 | + | 0 | 0 |
| | 36 | + | 0 | 12 (IgG) | + | 0 | 47 (IgG) |
| | 37 | + | 18 (IgG); 12 (IgM) | 0 | + | 61 (IgG) | 0 |
| | Mean (%) | 100 | 40 | 20 | 100 | 44 | 22 |

Tx: Transplantation; Tx aPL+ Siro+: Transplant recipients with Antiphospholipid Antibodies treated with sirolimus; Tx aPL+ Siro−: Transplant recipients with Antiphospholipid Antibodies without sirolimus; LA: Lupus Anticoagulant antibodies; NA: Not Available.

### DISCUSSION

By combining in vivo observations in humans with in vitro studies, the inventors were able to establish a pivotal role for the mTORC pathway in regulating the progression of vascular disease in APS. They demonstrate an activation of both mTORC1 and mTORC2 in the endothelial cells of APS patients with APS related chronic vascular remodelling. The direct causative role of APA in this setting is sustained by our finding that exposition of cultured human endothelial cells to APA elicits both mTORC1 and mTORC2 signalling. Taking advantage of the use of sirolimus as an immunosuppressive drug in transplanted patients, they demonstrated that mTORC activation acts as a growth-promoting factor that is instrumental in the constitution of the APS related chronic vascular lesions. Strikingly, they found that sirolimus therapy was associated with a preservation of the kidney architecture and function in transplanted patients with APA. As a whole, the present results identified a new mechanism of antibody mediated vascular injury and point to mTORC inhibition as a potential, molecular target, therapeutic strategy in APS.

Although many studies have been conducted to elucidate the molecular mechanisms leading to thrombosis during APS, the signalling pathways that allow antiphospholipid antibodies to vascular cells proliferation and progressive occlusion were unknown. The present study points to mTORC as the kinase that induces endothelial cells activation and proliferation in this setting. In fact, the inventors observed that mTORC is activated in endothelial cells in response to antiphospholipid antibodies binding consistent with previous in vitro studies reporting an activation of mTORC in cultured endothelial cells submitted to others type of antibodies such as anti-HLA antibodies⁴². More importantly, they demonstrated that, in human, the preclusion of such activation by sirolimus treatment led to prevention of vascular damage.

The present in vitro studies provided a direct link between APA and mTORC pathway activation. Interestingly, APA induced a rapid recruitment of mTORC1 and mTORC2 complexes. However, the mechanism by which mTORC is recruited by APA is not elucidated. APA have been demonstrated to promote thrombosis in part through ligation with the domain I of β2GPI on endothelial cell surface⁴³. This complex then cross-link many receptors including annexin A2, toll like receptor 4, calreticulin, apoER2 and nucleolin, leading to cell activation and thrombosis⁴⁴⁻⁴⁶. In the present model, after APA ligation to its cellular receptors, AKT is rapidly recruited to the membrane and phosphorylated on residue Thr³⁰⁸ in a PI3K dependant step as assessed by the impact of LY294002. Importantly, they observed, that endothelial mTORC2 could be sirolimus sensitive in some conditions. Indeed, a short time exposure of sirolimus led to a rapid blockage of mTORC1 but nor mTORC2. However, a more prolonged exposure led to a complete blockage of mTORC1 and mTORC2 pathways. This finding is in phase with recent results, obtain in vitro but also in vivo, where the mechanism of mTORC2 inhibition due to sirolimus is unclear, acting either by dissociating the mTORC2 complex or reducing RICTOR expression³²⁻⁴¹.

The present study revealed that, while mTORC activation is compartmentalized in endothelial cells, proliferation involved both, endothelial cells and VSMC. This observation supports that endothelial cells can lead to the release of a mitotic paracrine factor targeting VSMC, whom secretion is mTORC dependent. Endothelial cells play numerous physiological roles including the maintenance of vascular tone by release of molecules such as nitric oxide, endothelin-1 or prostacyclin⁴⁷, and normally inhibit VSMC proliferation^{48,49}. During mechanical injury, endothelial cells secrete many cytokines, such as platelet-derived growth factor (PDGF), that directly induce VSMC replication, deposition of extracellular matrix and may lead to the formation of a progressive obliterative neointima⁵⁰. Interestingly, similar findings have been done in the context of solid organ transplantation where endothelial injury is mediated by antibody⁵¹⁻⁵⁵. In this setting, anti-HLA antibodies are associated with chronic vascular remodelling and neointima information. This process has been linked to mTORC pathway activation^{42,56} and moreover to be everolimus sensitive, another mTORC inhibitor⁵⁷.

Despite systemic anticoagulation, the standard treatment of patients with APS, this regimen failed to fully prevent the recurrence of arterial accidents. The inventors provide here a new therapeutic option that is independent of haemostasis as they observed endothelial mTORC activation in spite of anticoagulation in patients with APA, and in vitro mTORC signalling elicited by APA in the absence of a functional coagulation cascade.

In conclusion, this work has established mTORC as a central axis in endothelial cell biology that modulates vascular injury mediated by APA. After kidney transplantation in patients with APA, disruption of this pathway prevents tissue damage, improved mGFR as well as allograft survival rate. Therefore, targeting mTORC pathway may represent an interesting strategy during APS or CAPS. Thus, prospective studies are needed to delineate the precise therapeutic field of mTORC inhibition in APS.

### REFERENCES

Throughout this application, various references describe the state of the art to which this invention pertains. The disclosures of these references are hereby incorporated by reference into the present disclosure.
1. Levine J S, Branch D W, Rauch J. The antiphospholipid syndrome. The New England journal of medicine 2002; 346:752-63.
2. Ruiz-Irastorza G, Khamashta M A, Hughes G R. Antiphospholipid antibodies and risk for recurrent vascular events. JAMA: the journal of the American Medical Association 2004; 291:2701; author reply 2-3.
3. Espinosa G, Cervera R. Morbidity and mortality in the antiphospholipid syndrome. Current opinion in pulmonary medicine 2009; 15:413-7.
4. Long B R, Leya F. The role of antiphospholipid syndrome in cardiovascular disease. Hematology/oncology clinics of North America 2008; 22:79-94, vi-vii.
5. Alarcon-Segovia D, Cardiel M H, Reyes E. Antiphospholipid arterial vasculopathy. The Journal of rheumatology 1989; 16:762-7.
6. Lie J T. Vasculopathy in the antiphospholipid syndrome: thrombosis or vasculitis, or both? The Journal of rheumatology 1989; 16:713-5.
7. Reyes E, Alarcon-Segovia D. Leg ulcers in the primary antiphospholipid syndrome. Report of a case with a peculiar proliferative small vessel vasculopathy. Clinical and experimental rheumatology 1991; 9:63-6.
8. Champion R H, Rook A. Cutaneous arteriolitis. Proceedings of the Royal Society of Medicine 1960; 53:568.
9. Deffer T A, Berger T G, Gelinas-Sorell D. Sneddon's syndrome. A case report. Journal of the American Academy of Dermatology 1987; 16:1084-7.
10. Macucci M, Dotti M T, Battistini S, et al. Primary antiphospholipid syndrome: two case reports, one with histological examination of skin, peripheral nerve and muscle. Acta neurologica 1994; 16:87-96.
11. Hughson M D, McCarty G A, Brumback R A. Spectrum of vascular pathology affecting patients with the antiphospholipid syndrome. Human pathology 1995; 26:716-24.
12. Nochy D, Daugas E, Droz D, et al. The intrarenal vascular lesions associated with primary antiphospholipid syndrome. J Am Soc Nephrol 1999; 10:507-18.
13. Daugas E, Nochy D, Huong D L, et al. Antiphospholipid syndrome nephropathy in systemic lupus erythematosus. J Am Soc Nephrol 2002; 13:42-52.
14. Tektonidou M G, Sotsiou F, Nakopoulou L, Vlachoyiannopoulos P G, Moutsopoulos H M. Antiphospholipid syndrome nephropathy in patients with systemic lupus erythematosus and antiphospholipid antibodies: prevalence, clinical associations, and long-term outcome. Arthritis and rheumatism 2004; 50:2569-79.
15. Canaud G, Bienaime F, Noel L H, et al. Severe vascular lesions and poor functional outcome in kidney transplant recipients with lupus anticoagulant antibodies. Am J Transplant 2010; 10:2051-60.
16. Stone J H, Amend W J, Criswell L A. Antiphospholipid antibody syndrome in renal transplantation: occurrence of clinical events in 96 consecutive patients with systemic lupus erythematosus. Am J Kidney Dis 1999; 34:1040-7.
17. Vaidya S, Gugliuzza K, Daller J A. Efficacy of anticoagulation therapy in end-stage renal disease patients with antiphospholipid antibody syndrome. Transplantation 2004; 77:1046-9.
18. Ducloux D, Pellet E, Fournier V, et al. Prevalence and clinical significance of antiphospholipid antibodies in renal transplant recipients. Transplantation 1999; 67:90-3.
19. Wagenknecht D R, Becker D G, LeFor W M, McIntyre J A. Antiphospholipid antibodies are a risk factor for early renal allograft failure. Transplantation 1999; 68:241-6.
20. Laplante M, Sabatini D M. mTOR signaling in growth control and disease. Cell 2012; 149:274-93.
21. Gallo R, Padurean A, Jayaraman T, et al. Inhibition of intimal thickening after balloon angioplasty in porcine coronary arteries by targeting regulators of the cell cycle. Circulation 1999; 99:2164-70.
22. Farb A, John M, Acampado E, Kolodgie F D, Prescott M F, Virmani R. Oral everolimus inhibits in-stent neointimal growth. Circulation 2002; 106:2379-84.
23. Sousa J E, Costa M A, Abizaid A C, et al. Sustained suppression of neointimal proliferation by sirolimus-eluting stents: one-year angiographic and intravascular ultrasound follow-up. Circulation 2001; 104:2007-11.
24. Finn A V, John M, Nakazawa G, et al. Differential healing after sirolimus, paclitaxel, and bare metal stent placement in combination with peroxisome proliferator-activator receptor gamma agonists: requirement for mTOR/Akt2 in PPARgamma activation. Circ Res 2009; 105:1003-12.
25. Stevens L A, Levey A S. Measured GFR as a confirmatory test for estimated GFR. J Am Soc Nephrol 2009; 20:2305-13.
26. Canaud G, Knebelmann B, Harris P C, et al. Therapeutic mTOR inhibition in autosomal dominant polycystic kidney disease: What is the appropriate serum level? Am J Transplant 2010; 10:1701-6.
27. Solez K, Colvin R B, Racusen L C, et al. Banff 07 classification of renal allograft pathology: updates and future directions. Am J Transplant 2008; 8:753-60.
28. Viau A, El Karoui K, Laouari D, et al. Lipocalin 2 is essential for chronic kidney disease progression in mice and humans. The Journal of clinical investigation 2010; 120:4065-76.
29. Janes M R, Limon J J, So L, et al. Effective and selective targeting of leukemia cells using a TORC1/2 kinase inhibitor. Nature medicine 2010; 16:205-13.
30. Manning B D, Cantley L C. AKT/PKB signaling: navigating downstream. Cell 2007; 129:1261-74.
31. Vlahos C J, Matter W F, Hui K Y, Brown R F. A specific inhibitor of phosphatidylinositol 3-kinase, 2-(4-morpholinyl)-8-phenyl-4H-1-benzopyran-4-one (LY294002). The Journal of biological chemistry 1994; 269:5241-8.
32. Sarbassov D D, Ali S M, Sengupta S, et al. Prolonged rapamycin treatment inhibits mTORC2 assembly and Akt/PKB. Molecular cell 2006; 22:159-68.
33. Lamming D W, Ye L, Katajisto P, et al. Rapamycin-induced insulin resistance is mediated by mTORC2 loss and uncoupled from longevity. Science (New York, N.Y. 2012; 335:1638-43.
34. Barilli A, Visigalli R, Sala R, et al. In human endothelial cells rapamycin causes mTORC2 inhibition and impairs cell viability and function. Cardiovasc Res 2008; 78:563-71.
35. Wang Y, Mikhailova M, Bose S, Pan C X, deVere White R W, Ghosh P M. Regulation of androgen receptor transcriptional activity by rapamycin in prostate cancer cell proliferation and survival. Oncogene 2008; 27:7106-17.
36. Zeng Z, Sarbassov dos D, Samudio I J, et al. Rapamycin derivatives reduce mTORC2 signaling and inhibit AKT activation in AML. Blood 2007; 109:3509-12.
37. Shu L, Houghton P J. The mTORC2 complex regulates terminal differentiation of C2C12 myoblasts. Molecular and cellular biology 2009; 29:4691-700.
38. Chen X G, Liu F, Song X F, et al. Rapamycin regulates Akt and ERK phosphorylation through mTORC1 and mTORC2 signaling pathways. Mol Carcinog 2010; 49:603-10.
39. Barlow A D, Xie J, Moore C E, et al. Rapamycin toxicity in MING cells and rat and human islets is mediated by the inhibition of mTOR complex 2 (mTORC2). Diabetologia 2012; 55:1355-65.
40. Letavernier E, Bruneval P, Vandermeersch S, et al. Sirolimus interacts with pathways essential for podocyte integrity. Nephrol Dial Transplant 2009; 24:630-8.
41. Vollenbroker B, George B, Wolfgart M, Saleem M A, Pavenstadt H, Weide T. mTOR regulates expression of slit diaphragm proteins and cytoskeleton structure in podocytes. Am J Physiol Renal Physiol 2009; 296:F418-26.
42. Lepin E J, Zhang Q, Zhang X, et al. Phosphorylated S6 ribosomal protein: a novel biomarker of antibody-mediated rejection in heart allografts. Am J Transplant 2006; 6:1560-71.
43. de Laat B, Derksen R H, van Lummel M, Pennings M T, de Groot P G. Pathogenic anti-beta2-glycoprotein I antibodies recognize domain I of beta2-glycoprotein I only after a conformational change. Blood 2006; 107:1916-24.
44. Allen K L, Hamik A, Jain M K, McCrae K R. Endothelial cell activation by antiphospholipid antibodies is modulated by Kruppel-like transcription factors. Blood 2011; 117:6383-91.
45. Allen K L, Fonseca F V, Betapudi V, Willard B, Zhang J, McCrae K R. A novel pathway for human endothelial cell activation by antiphospholipid/anti-beta2 glycoprotein I antibodies. Blood 2012; 119:884-93.
46. Ramesh S, Morrell C N, Tarango C, et al. Antiphospholipid antibodies promote leukocyte-endothelial cell adhesion and thrombosis in mice by antagonizing eNOS via beta2GPI and apoER2. The Journal of clinical investigation 2011; 121:120-31.
47. Ross R. The pathogenesis of atherosclerosis: a perspective for the 1990s. Nature 1993; 362:801-9.
48. Peiro C, Redondo J, Rodriguez-Martinez M A, Angulo J, Marin J, Sanchez-Ferrer C F. Influence of endothelium on cultured vascular smooth muscle cell proliferation. Hypertension 1995; 25:748-51.
49. Milliat F, Francois A, Isoir M, et al. Influence of endothelial cells on vascular smooth muscle cells phenotype after irradiation: implication in radiation-induced vascular damages. The American journal of pathology 2006; 169:1484-95.
50. Ferns G A, Raines E W, Sprugel K H, Motani A S, Reidy M A, Ross R. Inhibition of neointimal smooth muscle accumulation after angioplasty by an antibody to PDGF. Science (New York, N.Y. 1991; 253:1129-32.
51. Galvani S, Auge N, Calise D, et al. HLA class I antibodies provoke graft arteriosclerosis in human arteries transplanted into SCID/beige mice. Am J Transplant 2009; 9:2607-14.
52. Amano J, Ishiyama S, Nishikawa T, et al. Proliferation of smooth muscle cells in acute allograft vascular rejection. The Journal of thoracic and cardiovascular surgery 1997; 113:19-25.
53. Shi C, Feinberg M W, Zhang D, et al. Donor M H C and adhesion molecules in transplant arteriosclerosis. The Journal of clinical investigation 1999; 103:469-74.
54. Geraghty J G, Stoltenberg R L, Sollinger H W, Hullett D A. Vascular smooth muscle cells and neointimal hyperplasia in chronic transplant rejection. Transplantation 1996; 62:502-9.
55. Grimm P C, Nickerson P, Jeffery J, et al. Neointimal and tubulointerstitial infiltration by recipient mesenchymal cells in chronic renal-allograft rejection. The New England journal of medicine 2001; 345:93-7.
56. Eisen H J, Tuzcu E M, Dorent R, et al. Everolimus for the prevention of allograft rejection and vasculopathy in cardiac-transplant recipients. The New England journal of medicine 2003; 349:847-58.
57. Boersema M, Katta K, Rienstra H, et al. Local medial microenvironment directs phenotypic modulation of smooth muscle cells after experimental renal transplantation. Am J Transplant 2012; 12:1429-40.
58. Viard J P, Amoura Z, Bach J F. Association of anti-beta 2 glycoprotein I antibodies with lupus-type circulating anticoagulant and thrombosis in systemic lupus erythematosus. Am J Med 1992; 93:181-6.

1. A method of preventing or treating antiphospholip syndrome (APS)-related vascular lesions and/or inhibiting or alleviating symptoms of endothelial mTORC activation triggered by antiphospholipid antibodies (APA) in a patient in need thereof, comprising
administering to said patient a therapeutically effective amount of a phosphatidylinositide 3-kinase (PI3K) AKT-mammalian target of rapamycin (mTOR) pathway inhibitor.

2. The method according to claim 1, wherein the APS-related vascular lesions are APS-nephropathy (APSN).

3. (canceled)

4. The method according to claim 1, wherein the patient in need thereof is affected with a primary APS, a secondary APS, a catastrophic APS (CAPS) or is a transplant recipient with APA.

5. The method according to claim 4, wherein the transplant recipient with APA is a kidney transplant recipient.

6. The method according to claim 1, wherein the PI3K-AKT-mTOR pathway inhibitor is a PI3K inhibitor.

7. The method according to claim 6, wherein the PI3K inhibitor is selected from the group consisting of LY2940002, SF1126, PI103, GDC 0941, XL765, XL147, BGT226, BEZ235 and an inhibitor of PI3K gene expression.

8. The method according to claim 1, wherein the PI3K-AKT-mTOR pathway inhibitor is an AKT inhibitor.

9. The method according to claim 8, wherein the AKT inhibitor is selected from the group consisting of Perifosine, XL418, GSK690693, AT13148, A-443654 and an inhibitor of AKT gene expression.

10. The method according to claim 1, wherein the PI3K-AKT-mTOR pathway inhibitor is a mTOR inhibitor.

11. The method according to claim 8, wherein the mTOR inhibitor is selected from the group consisting of rapamycin (sirolimus), temsirolimus, deforolimus, everolimus, tacrolimus, a rapamycin analog or derivative thereof, torin1, PP242 and an inhibitor of a member of mTOR complex gene expression.

12. The method according to claim 10, wherein the mTOR inhibitor is rapamycin (sirolimus).

13. A pharmaceutical composition comprising a PI3K AKT mTOR pathway inhibitor and a pharmaceutically acceptable carrier.

14. The pharmaceutical composition according to claim 13 further comprising an additional therapeutic agent.

15. A kit comprising at least two PI3K-AKT-mTOR pathway inhibitors as a combined preparation for simultaneous, separate or sequential administration.

16. The kit according to claim 15, wherein said at least two PI3K-AKT-mTOR pathway inhibitors are (a) a PI3K inhibitor and an AKT inhibitor; (b) a PI3K inhibitor and a mTOR inhibitor; (c) an AKT inhibitor and a mTOR inhibitor; and (d) a PI3K inhibitor, an AKT inhibitor and a mTOR inhibitor.

17. A method of preventing graft rejection and/or preserving graft function in a patient in need thereof, comprising
administering to said patient a therapeutically effective amount of a phosphatidylinositide 3-kinase (PI3K) AKT-mammalian target of rapamycin (mTOR) pathway inhibitor.

18. The method according to claim 17, wherein the patient in need thereof is a kidney transplant recipient with APA.
